(19) United States
(12) Patent Application Publication
(10) Pub. No.: US 2022/0122716 A1
(43) Pub. Date: Apr. 21, 2022

(54) ARTIFICIAL INTELLIGENCE-BASED, COGNITIVE SYSTEM AND METHOD FOR MODIFYING SUBCONSCIOUS BRAIN HABITUALIZATION

(71) Applicant: Metabolic and Cardiovascular Institute of Florida, LLC, West Palm Beach, FL (US)

(72) Inventor: Samuel Sadow, West Palm Beach, FL (US)

(21) Appl. No.: 17/505,237

(22) Filed: Oct. 19, 2021

Related U.S. Application Data
(60) Provisional application No. 63/093,618, filed on Oct. 19, 2020.

(51) Int. Cl. G16H 20/70; A61B 5/145; A61B 5/11; A61B 5/00; A61B 5/0205; A61B 5/318; G09B 5/02; G09B 5/06

(52) U.S. Cl. CPC G16H 20/70; A61B 5/14532; A61B 5/14546; A61B 5/1118; A61B 5/4806; G09B 19/0092; A61B 5/4836; A61B 5/318; G09B 5/02; G09B 5/065; A61B 5/0205

(57) ABSTRACT

A cognitive behavioral system and personalized methods using artificial intelligence (AI) for modifying subconscious brain habitualization relating to weight loss or weight loss maintenance. The systems and methods center upon subconscious reprogramming of individuals in order to more successfully obtain long-term weight loss and/or weight loss maintenance. The subconscious reprogramming includes the unlearning of old and established behavior which has led the user to engage in unhealthy habits and adiposity based chronic disease as it relates to weight loss/gain.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/093,618, entitled, “AN ARTIFICIAL INTELLIGENCE-BASED, COGNITIVE SYSTEM AND METHOD FOR MODIFYING SUBCONSCIOUS BRAIN HABITUALIZATION”, filed on Oct. 19, 2020. The contents of the above referenced application are incorporated herein by reference in their entirety.

### FIELD OF THE INVENTION

The present invention relates to health care related systems and methods; more particularly, to a system and method of employing subconscious brain habitualization along with cognitive intensive nutrition, lifestyle and behavior therapy to aid individuals to unlearn unhealthy habits related to food related behavior, lifestyle, and nutrition therapy as a mechanism of weight loss and weight loss maintenance.

### BACKGROUND OF THE INVENTION

Adiposity-based Chronic Disease (ABCD) is responsible for an estimated $190 billion per year, or approximately 21% of the healthcare related costs in the U.S., see webpage, ncbi.nim.nih.gov. Approximately 10.5% of people in the U.S. have obesity and Type 2 diabetes (T2DM), costing an average of $16,750 per person per year. The economic cost of diabetes in the U.S. in 2017 was $327 billion, of which $237 billion was direct and $90 billion was indirect costs, see Economic Costs of Diabetes in the U.S. in 2017. Diabetes Care 2018 March; dci180007. Following the American Diabetes Association's Diabetes Prevention (DPP) multi-center trials for obese, pre-diabetes in 2002 (The Diabetes Prevention Program (DPP) Research Group., the Diabetes Prevention Program (DPP): description of lifestyle intervention. Diabetes Care. 2002 December; 25(12):2165-2171), the U.S. Preventive Services Taskforce (PSTF) in 2018 issued the Standard of Care recommendations for obesity. The first PSTF recommendation for obesity was intensive, cognitive, multidisciplinary, medical intervention for the responsible three (3) unhealthy habits: behavior, lifestyle, and nutrition therapy, see A three-dimensional program for the treatment of obesity, Richard B. Stuart, Behavior Research and Therapy, Aug. 3, 1971, Vol. 9., Issue 3, pages 177-186.

According to Stuart's cognitive behavioral theory (CBT), obesity starts in childhood by unhealthy, learned habits, which can be “unlearned” and new, healthy habits learned. Any of the three (3) unhealthy habits make it challenging for people to lose significant weight and keep weight off due to deep learning in the subconscious brain. The present invention addresses these findings by providing a unique, efficient, and predictable weight loss system and methods thereof.

### SUMMARY OF THE INVENTION

A self-contained, end-to-end cognitive system using Artificial Intelligence (AI) and method for modifying and verifying subconscious brain habitualization. The systems and methods provide for subconscious brain remodeling for habitualization, which is critical to long-term successful unlearning. The present invention relates to systems and methods which provide unique solutions for significant weight loss and/or weight loss maintenance, such as for adiposity based chronic disease (ABCD) remission for optimal health, substance abuse, alcoholism, and population health. The systems and methods are directed towards machine and deep learning for subconscious reprogramming of individuals in order to more successfully and cost efficiently obtain weight loss and/or weight loss maintenance for virtual chronic disease intervention for remission, health care cost savings, improved outcomes, improved satisfaction, reduced staff burden, and data collection. The subconscious reprogramming includes the unlearning of old and established behavior which has led an individual to engage in, or adopt, unhealthy habits as it relates to weight loss/gain. Once the unlearning of old and established behavior is complete, the systems and methods in accordance with Applicant's invention provide the user the ability to learn new, healthy habits which can assist the individual in weight loss and weight loss maintenance.

The systems and methods provide personal, structured, self-learning framework employing clinical and patient reported answers, as well as key word targeting associated with the three (3) components of CBT, i.e. behavior, lifestyle, and nutrition therapy to: a) teach participants as they go, and b) build up and deliver AI-based responses, and c) measure compliance and increase it. The system and methods aid the user in removing unhealthy habits in order to reprogram the user's behavior, such as by reprogramming, i.e. relearning of the user's subconscious brain's ability to seamlessly and permanently make healthy choices as it relates to healthy food related habits in order to lose weight and then maintain the weight loss.

The method of weight loss system, using one or more components of the weight loss system, is driven by one or more, in any combination, of: 1) a platform/software program that implements a Cognitive Behavioral Therapy algorithm that utilizes various data about the user, and is configured to provide various feedback in the form of personalized user messages relating to task completions, response to clinical progress, and motivation, i.e. congratulatory or non-congratulatory messages; 2) databases for receiving and storing data sets related to the user, such as compliance data (is the user sticking to the program, such as have they answered the daily questionnaire or survey, or watched a podcast), clinical data, clinical progress data, patient reported data (i.e. daily questionnaire or survey; 3) one or more health related, physical parameters measurement devices designed to monitor, measure, and obtain data associated with one or more health related physical attributes of the user; 4) use of video or text through, for example, podcasts; 5) key word messaging; 6) meditation; 7) biofeedback loops, i.e. messaging the user based on data obtained or response to a message that was already sent to the user; and 8) continuously educating and expanding personal algorithms for increased functionality.

Another illustrative embodiment of the invention provides a non-transitory computer readable medium having computer readable instructions embodied thereon for an automated cognitive behavioral system for modifying subconscious brain habitualization, wherein, when executed by at least one processor of an electronic device used by said individual requiring modifying subconscious brain habitualization, the computer-executable instructions cause at least one processor to at least perform operations to accomplish such functionality.

Another illustrative embodiment of the invention provides a system for electronically modifying subconscious brain habitualization comprising an electronic device, a processor operable to execute instructions, and a data storage medium or database for storing instructions and or data which, when executed by the processor, cause the processor to accomplish any functionality described herein.

Accordingly, it is an objective of the invention to provide unique weight loss systems using an automated cognitive behavioral system for modifying subconscious brain habitualization using artificial intelligence (AI) to evaluate and enhance effectiveness over time.

It is a further objective of the invention to provide unique methods for achieving weight loss using an automated cognitive behavioral method and for modifying subconscious brain habitualization, the automation providing rapid response, consistency, and freeing the physician from repetitive interactions.

It is yet another objective of the invention to provide unique weight loss maintenance systems using an automated cognitive behavioral system for modifying subconscious brain habitualization.

It is a still further objective of the invention to provide unique methods for maintaining weight loss using a cognitive behavioral system for modifying subconscious brain habitualization.

It is a further objective of the invention to provide systems and methods for weight loss and/or weight loss maintenance targeting the unhealthy habits of behavior, lifestyle, and nutrition therapy, in order to provide a user with the ability to make healthy choices.

It is yet another objective of the present invention to provide systems and methods for weight loss and/or weight loss maintenance targeting the unhealthy habits of behavior, lifestyle, and nutrition therapy, in order to provide a user with the ability to make healthy choices, using Machine learning to monitor and enhance the effectiveness of these systems and methods.

It is a still further objective of the invention to provide systems and methods for weight loss and/or weight loss maintenance targeting the unhealthy habits of behavior, lifestyle, and nutrition therapy in order to provide a user with the ability to make healthy choices, using Machine learning and deep learning to monitor and enhance the effectiveness of this process.

It is a further objective of the present invention to provide systems and methods for weight loss and/or weight loss maintenance targeting the unhealthy habits of behavior, lifestyle, and nutrition therapy in order to provide a user with the ability to make healthy choices which utilize biofeedback messaging, thus helping to reinforce subconscious recalibration.

It is yet another objective of the present invention to provide systems and methods for weight loss and/or weight loss maintenance targeting the unhealthy habits of behavior, lifestyle, and nutrition therapy in order to provide a user with the ability to make healthy choices which utilize patient feedback via questionnaires.

It is a still further objective of the invention to provide systems and methods for weight loss and/or weight loss maintenance targeting the unhealthy habits of behavior, lifestyle, and nutrition therapy in order to provide a user with the ability to make healthy choices which utilize meditation.

It is a still further objective of the invention to provide systems and methods for weight loss and/or weight loss maintenance which utilize self-learning methods to develop behavioral strategies useful for such weight loss and/or weight loss maintenance.

It is a still further objective of the invention to provide systems and methods which utilize predictive modeling of the results of subconscious recalibration, compliance, and outcomes which will be documented using smart questionnaires.

It is a still further objective of the invention to provide systems and methods which utilize Artificial neural networks for deep learning to identify clusters of characteristics that identify patients likely to succeed and guide improvements to provide success for a greater variety of patients and situations, using unsupervised learning techniques.

It is a still further objective of the invention to provide systems and methods applicable to situations in which individuals require establishment of new, healthy habits for other non-obesity-related conditions, such as substance abuse recovery, depression, psychoneurosis, and alcoholism.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a schematic diagram of an embodiment of the hardware or software components associated with a schematic illustration of a cognitive behavioral system for modifying subconscious brain habitualization relating to weight loss or weight loss maintenance;

FIG. 2 is a schematic diagram of an embodiment of the hardware and software components associated with a schematic illustration of a cognitive behavioral system for modifying subconscious brain habitualization relating to weight loss or weight loss maintenance;

FIG. 3 is an illustrate embodiment of a cognitive behavioral method for modifying subconscious brain habitualization relating to weight loss or weight loss maintenance;

FIG. 4 is a schematic diagram of the interactions of various components of the weight loss system.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention relates to systems and methods using a cognitive behavioral system for modifying subconscious brain habitualization. The present systems and methods provide an end-to-end ecosystem for reducing healthcare costs, improving outcomes, and improving satisfaction of individuals with ABCD. Significant (>5-8%) weight loss is necessary for chronic disease remission, however weight loss without a formal program designed to “un-learn” obesogenic habits from childhood is destined to long-term failure in a majority of individuals. Central to long-term success is re-programming of the subconscious. Such systems and methods may provide, for example, unique solutions for weight loss and/or weight loss maintenance. The systems and methods center upon cognitive subconscious reprogramming of individuals to obtain weight loss and/or weight loss maintenance more successfully. The subconscious reprogramming includes the unlearning of old and established behavior which has led the individual to unhealthy habits as it relates to weight loss/gain. Simultaneous with unlearning of old and established behavior, the systems and methods in accordance with Applicant's invention provide the user with learning of new healthy habits which can assist the individual in weight loss and weight loss maintenance. While described as part of a weight loss and weight loss maintenance system, the present invention may be adapted to provide behavior adjustment associated with other medical conditions to provide mechanisms for improving other medical conditions not related to Type 2 diabetes (T2DM).

The core of the systems, process, or methods using the cognitive behavioral system for modifying subconscious brain habitualization is the interactions between the patient/user and the system. Such interactions can take place using a web site, smart phone application, or similar. These interactions are of several forms: the system (and methods) contains a scheduler that generates prompt(s) sent to the patient/user on a regular basis (such as two times a day, three times a day, every other day), which consist of questions, quizzes, links to podcasts, etc. Some of the interactions are designed to be one way, i.e., the system sends the messages and with no patient/user input or action response is required. Other patient/user interactions will result in a reaction from the patient/user, such as clicking on a link, replying to a question, etc. The system will store the details of these interactions in the database, including when the prompt was sent, when (or whether) the patient replied, when that was, and what the time between the prompt and reply was. The patient can also initiate interactions with the system, including self-reported medical statistics, queries, and information about their mood and motivations. These will also be stored in the database. There can also be automated medical reporting devices/physical parameter measurement devices (glucometers, scales, etc.) that send data to the system on the patient/user's behalf without need for the patient/user to interact or initiate the exchange. This is “adaptive intelligence”.

The data in the database can be analyzed to derive measures on the patients' participation, engagement, compliance and success. These metrics can also be stored in the database. A As used above, “participation” and “engagement” can be used synonymously and may be defined as the act of completing, or attempting to complete, a particular task. As used above, “compliance” may be defined as completing and submitting a certain number of quiz answers to the Daily Therapy database. The total number of quiz answer submissions above a predetermined number, preferably the total of 60 quiz, or completing at least 2 quizzes/week out of 12 weeks. Patients/users completing and submitting at least 25 out if 60 quizzes always tend to be engaged and motivated to lose enough weight to enter T2DM remission.

An artificial intelligent (AI) system can then periodically process the collected data in order to form associations between parameters of the prompts and feedback (time, which one chosen, current patient situation, etc.) and their results (quickness of response, patient's mood and compliance, improvement in medical parameters) and use these associations to adjust weights in the algorithm to gradually identify and prefer the more effective interactions, i.e. provide for supervised learning. The neural networks utilize an array of weights that map input data to an output parameter. By adjusting these weights, it is possible to fine tune which inputs favor which results. The notion is that by evaluating the success of these results and assigning a “score”, that score can be used to adjust the weights dynamically. The scores may be derived from what happens after feedback is sent or delivered. If the patient/user's enthusiasm/compliance/cooperation/statistics has improved, that would be a positive score. If patient/user's enthusiasm/compliance/cooperation/statistics get worse, that would be a negative score. A positive score for a given set of inputs and output will tend to increase the weights associated with that linkage. A negative score will decrease the weights. After multiple iterations, the network is programmed to “learn” the preferred outputs for given patterns of input. One type of feedback that may include total weight loss %. For example, a goal of 20% weight loss overall, and 1-2% per week. Alternatively, or in combination, any of the physical parameter measurements, i.e. glucose or heart measurements may be used as well.

As used herein, the process of “supervised learning”, is an artificial intelligence process where inputs and outputs are correlated with the resulting feedback to adjust weights in a neural network to train the network to optimize for improved feedback by adjusting its outputs. Effectiveness could depend on subtle ways on many of the possible variable parameters in the interactions. The interaction chosen is an obvious one: some patients might be more motivated by messages such as “Great job!” and others by “Your mother (her name) would be proud of you.” Similarly, there might be a novelty/familiarity effect: the same motivational message might be less effective if it is chosen too frequently. Other parameters could be time of day: for example, sending a question early in the day might be effective for some patients. Other, more subtle determinations are possible as well: perhaps a certain phrase would be more effective for a particular situation, but less so for others.

In order to provide the algorithms with the raw materials to derive the most useful correlations, they will be supplied with as much information as possible. Not only the input data itself (such as blood glucose), but derived data (such as blood glucose trends), and even analytical metadata [such as another algorithm, see below under “Unsupervised Learning”) identifying a cluster of information as indicating the patient/user is in a good mood or likely to be considering an unhealthy choice]. One strength of neural networks in accordance with embodiments sf the system and methods is their ability to build weights and associations between non-obvious relationships in the data while discounting uncorrelated noise. With random feedback or noise, the weights could be adjusted up and down approximately equally, averaging out to no particular change. With correlated results (a specific output producing a positive score in general), the weights associated with it will accumulate. This removes the necessity for administrators to identify in advance all the possible relationships and simply provide the algorithm with a rich set of data on which to operate.

Initially, the algorithms will be provided some basic structure to the interactions, so the algorithms will not be entirely random, but based on existing, tested, clinical practice. There is an expected order and flow to the questions, and some straightforward parameters (like not repeating the same motivation too often) will be coded into the system and methods initially.

Another branch of artificial intelligence concerns automatically finding groups or clusters in data, and referred to as “unsupervised learning”. Unsupervised learning does not depend on having feedback available. It simply analyzes data sets and groups items together based on their association and similarity. Such sort of analysis may be done on the aggregate data to identify clusters of behavior that may be predictive of patient success or identify different situations (eagerness, crisis, temptation, etc.). The results of this clustering may be provided as an additional input to the selection algorithm which can in turn find correlations between these clusters and effective feedback. For example, perhaps one set of conditions such as blood glucose and weight are not declining, could be associated with a loss of motivation to continue on the program, and the learning algorithm could associate this with particular forms of encouragement, perhaps “You can do this!” or “Marines, Don't give up! would be particularly effective at such junctures. In certain embodiments of the system and methods, we are only using unsupervised learning may be used to group clusters of related parameters, for identifying specific situations that may respond differently to interactions.

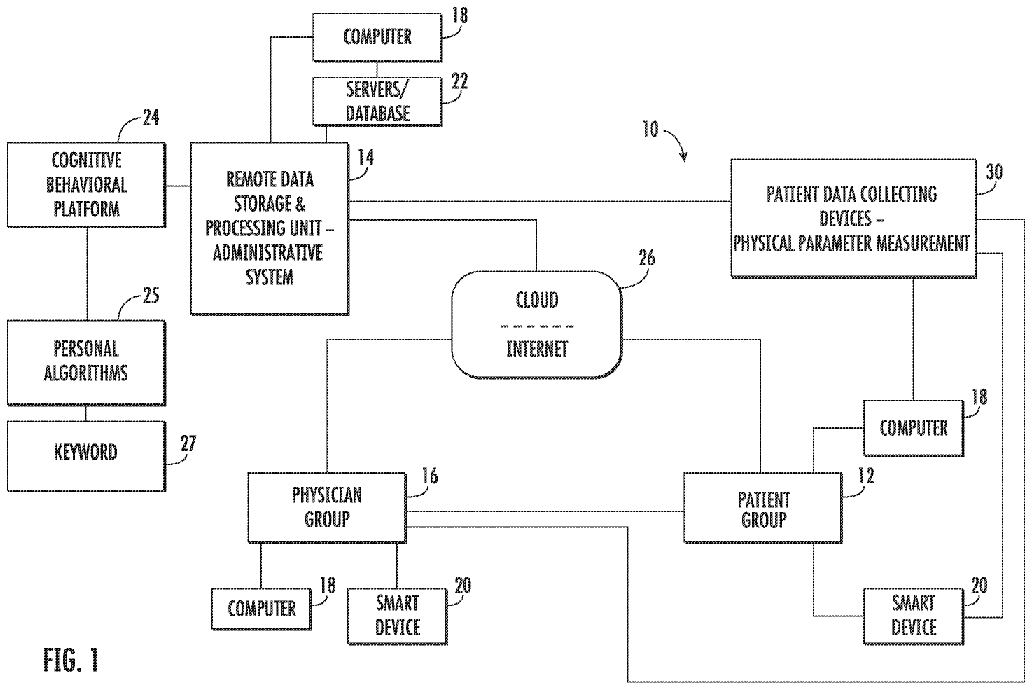

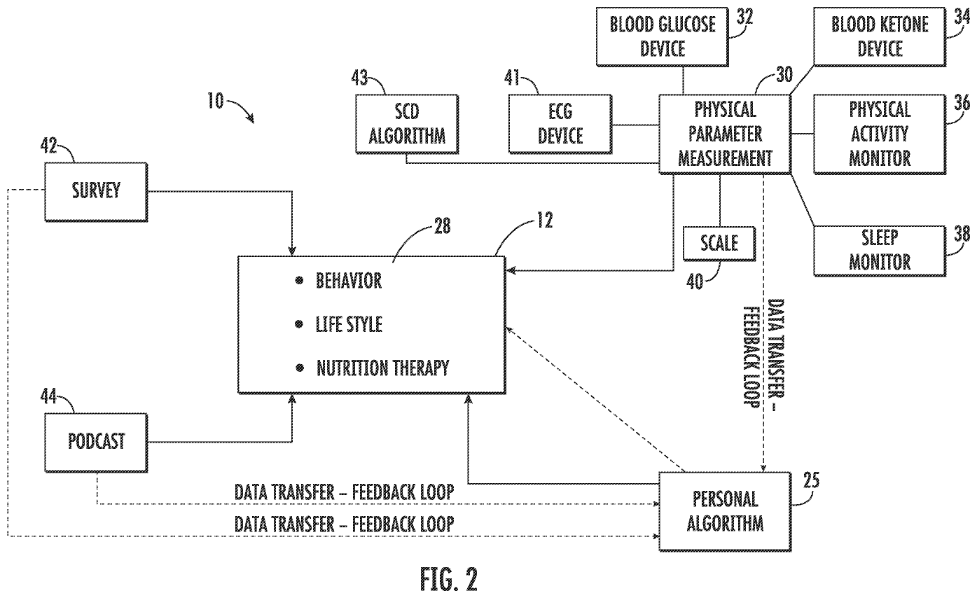

Referring to FIGS. 1 and 2, a schematic illustration of a cognitive behavioral system for modifying subconscious brain habitualization relating to weight loss or weight loss maintenance, referred to generally as a weight loss system 10, is illustrated. The weight loss system 10 is designed for subconscious reprogramming of an individual's pre-learned, unhealthy habits relating to behavior, lifestyle, and nutrition therapy that make weight loss and weight loss maintenance difficult. FIG. 1 illustrates one or more hardware and/or software components associated with system communication and data storage. FIG. 2 illustrates one or more hardware and/or software components associated with the analysis functionality. Each of the weight loss system 10 components, individually or in any combination, may be used to provide methods of weight loss, weight loss maintenance, or weight loss and weight loss maintenance.

The weight loss system 10 is designed to allow for electronic communication between a user, such as a patient group (when used to indicate a single user, referred to as user 12 or patient/user 12), and one or more data storage and processing unit(s) 14, which may be part of an administrative system which drives functional aspects of the system and methods described herein. A monitoring group, such as an employer, physician group, or third party administrator (TPA) 16, may also be functionally connected to the patient group 12, the data storage and processing unit 14, or the patient group 12 and the data storage and processing unit 14. While the patient group 12 is preferably configured for conferring individual use or functionality, the patient group 12 could also be configured as two or more patients grouped together for group functionality. The physician group 16 is preferably configured for individual physician use, but could also be configured for multiple physician use. The use of patient/user 12, the data storage and processing unit/administrator 14, and the employer, physician group 16 may also include portal systems for communication between the groups and the necessary functions. i.e. sending or receiving information, storing data, processing data, or sending/receiving texts, of the systems and methods described herein.

Both the patient group 12 and the physician group 16 may communicate with each other electronically using, for example, a computer 18 or smart device 20, such as a smart cell phone or computer tablet, such as an IPAD. Both the patient group 12 and the physician group 16 may also communicate with the data storage and processing unit 14 electronically using, for example, a computer 18 or smart device 20, such as a smart cell phone or computer tablet, such as an IPAD. The one or more data storage and processing unit(s) 14 are shown as independent components or units. However, the one or more data storage and processing unit(s) 14 may be part of the computers 18 or smart devices 20 associated with the patient group 12 and/or the physician group 16.

Regardless of the type of device used, the weight loss system 10 is configured to use electronic devices for communication and, or data collection purposes, allowing the patient group 12 to provide, automatically or via the user 12, sending of data to the data storage and processing unit 14. While shown as a separate component, the data storage and processing unit 14 may be part of the physician group 16. Accordingly, the weight loss system 10 includes the necessary hardware and software to accomplish its intended functionality. The computer 18 or smart device 20 may contain one or more of: Central Processing Unit (CPU), memory, storage, input control, modem, network interface, and the necessary software specifically designed to provide analysis and determinations of treatment options. Additionally, the one or more data storage and processing units 14 may include one or more computer(s) 18 or server(s) 22 configured for data collection, data storage and/or data analysis. The one or more data storage and processing units 14 may include the necessary software to generate the necessary automated cognitive behavioral platform, using artificial intelligence, 24 and the one or more algorithms 25, such as personal algorithms (based on specific user/patient inputs or actions or messages for a specific user) or general algorithms, stored within a memory for correlating data from the analysis portion associated with the weight loss system 10 to create an individualized (i.e. unique to a single user) user based response. Each of the individual components of the weight loss system 10 may be designed to communicate and/or be accessible with each other through the internet 26 (cloud-based communications). The weight loss system 10 may utilize unique “key words” 27 for algorithm modeling. Illustrative examples of key words may include a user's: nickname, mother, father, wife, grandchild's names, high school, best friend, favorite place, 1st car, favorite sports team, hobby, favorite unhealthy food, favorite alcoholic beverage. The key words 27, such as the personal names, places, events, are messaged to the patient/user in order to positively reinforce the importance of the patient/user's attitudes, results, responses, and compliance.

The cognitive behavioral system 10 may be designed to help individual patients, particularly those suffering from Type 2 diabetes (T2DM), with a patient modification mechanism, i.e. weight loss system. While the cognitive behavioral system 10 is illustrated for use in alleviating issues related to T2DM, the cognitive behavioral system 10 may be configured to alleviate other issues related to other diseases. Referring to FIG. 2, the cognitive behavioral system 10 may comprise one or more hardware and/or software components associated with the analysis functionality. Each of the components may drive the cognitive behavioral system 10 functionality to provide users 12 suffering from T2DM with a mechanism to “unlearn” unhealthy, previously learned habits 28 as it relates to food related behavior, lifestyle, and nutrition therapy. Accordingly, the cognitive behavioral system 10 may contain one or more health physical parameters measurement devices 30 that are designed to help recalibrate the participant's subconscious using biofeedback to reinforce their importance. The one or more health physical parameters measurement devices are designed to measure different physiological related activities or clinical measurements, including one or more parameters that effect weight loss/gain or weight loss maintenance. The data obtained may be electronically sent to, stored in, analyzed, and/or manipulated by the one or more data storage and processing units 14, or other computer systems described herein.

The cognitive behavioral system 10 may include a blood glucose device 32, such as the Keto Mojo blood glucometer, configured to measure the user's 12 blood glucose levels. The blood glucose device 32 is preferably electronically/functionally coupled to the one or more data storage and processing units 14 so that blood glucose levels measured are recorded and stored over time. The blood glucose device 32 may separately be electronically/functionally coupled to the user's computer 18 or smart device 20, and/or to the physician group's 16 computer 18 or smart device 20.

The cognitive behavioral system 10 may include a blood ketone device 34, such as the Keto Mojo blood ketone meter, configured to measure the user's 12 blood ketone levels necessary for initiating or maintaining or fat burning in the liver. The blood ketone device 34 is preferably electronically/functionally coupled to the one or more data storage and processing units 14 so that blood ketone levels measured are recorded and stored over time. The blood ketone device 34 may separately be electronically/functionally coupled to the user's 12 computer 18 or smart device 20, and/or to the physician group's 16 computer 18 or smart device 20.

The cognitive behavioral system 10 may include a physical activity monitoring device 36, such as the NOKIA Sleep & Activity Tracker, configured to track the user's 12 physical activities, such as the distances the user 12 runs, walks, swims, or the number of steps taken in a time period. The physical activity monitoring device 36 is preferably electronically/functionally coupled to the one or more data storage and processing units 14 so that physical activity related levels measured are recorded and stored over time. The physical activity monitoring device 36 may separately be electronically/functionally coupled to the user's 12 computer 18 or smart device 20, and/or to the physician group's 16 computer 18 or smart device 20.

The cognitive behavioral system 10 may include a sleep monitoring device 38, such as the NOKIA Sleep & Activity Tracker, configured to measure the user's 12 sleep activities, such as sleep stages and cycles, brain waves, breathing, heart rate, body movement, leg movement, eye movement, blood oxygen levels, position of the body during the sleep period, etc. The sleep monitoring device 38, is preferably electronically/functionally coupled to the one or more data storage and processing units 14 so that sleep related activities measured are recorded and stored over time. The physical activity monitoring device 36 may separately be electronically/functionally coupled to the user's 12 computer 18 or smart device 20, and/or to the physician group's 16 computer 18 or smart device 20.

The cognitive behavioral system 10 may include a body scale 40, such as the NOKIA Body Scale using WIFI, configured to measure the user's 12 body weight or body mass index. The scale 40 is preferably electronically/functionally coupled to the one or more data storage and processing units 14 so body weight or body mass index measured is recorded and stored over time. The scale 40 may separately be electronically/functionally coupled to the user's 12 computer 18 or smart device 20, and/or to the physician group's 16 computer 18 or smart device 20.

Obesity and T2DM increase the incidence of Sudden Cardiac Death (SCD). In order to motivate subconscious recalibration and compliance by self-learning, the weight loss system 10 may include a portable ECG device 41, such as KardiaMobile (Alivecor), directed to a proprietary algorithm for predicting SCD in one (1) second.

In certain embodiments of the cognitive behavioral system 10, one or more of the physical parameter measurement devices 30, such as the blood glucometer 32, the blood ketone meter 34, the physical activity 36 and sleep monitor 38, electronic scale 40, ECG 41 and sudden cardiac algorithm (SCD) 43, may be operated wirelessly, through for example BLUETOOTH technology.

The cognitive behavioral system 10 may utilize other devices/tools to obtain data on the user 12. For example, a data questionnaire(s) or survey(s) 42 may be generated and submitted, preferably electronically through a computer 18 or smart device 20, to the user 12. The data obtained, i.e. the answers to the data questionnaires or surveys 42, may be stored and analyzed by the one or more data storage and processing units 14. The data questionnaire(s) or survey(s) 42 may be in the form of one or more prescreened questions that are designed to document and help identify, predict, and identify factors promoting subconscious recalibration and contributing to compliance, satisfaction, weight loss and maintenance, and reduced absenteeism and presenteeism. The cognitive behavioral system 10 may include one or more podcasts 44. The podcasts 44 are videos that may comprise content related to behavioral strategies, nutritional strategies, and lifestyle strategies, all designed to aid the user 12 in the development of new habits relating to weight loss and weight maintenance. Based on the response to the data questionnaire(s) or survey(s) 42, the user, via an administrator 14 may be sent one or more text messages.

In certain embodiments of the cognitive behavioral system 10 or methods, a number of, such as sixty (60), behavioral, nutrition, and lifestyle strategy podcasts and parallel interactive quizzes, (“Daily Therapy”), are automatically sent five days per week for 12 weeks from our Microsoft SQL server, interoperable care delivery platform and database. Each participant has a personal dashboard and message board from an automated, AI-based health coach with human augmentation.

In certain embodiments of the cognitive behavioral system 10 or methods, participants read each Daily Therapy and Quiz session for 12 weeks, complete quizzes, and submit quiz answers. Each quiz answer is answered according to the participant's personal algorithm. Each completed quiz is entered into the participant's compliance database and triggers an AI-based motivational response or correction. Human augmentation may also respond.

In certain embodiments of the cognitive behavioral system 10 or methods, submission of quiz answers, blood glucose, blood ketones, physical activity, sleep, and weight loss will be entered into databases and used to calculate compliance and predictive modeling.

The system is designed and scalable for businesses to lower healthcare costs, improve outcomes, worker satisfaction, and staff efficiency. Each value is measured and separately entered.

In certain embodiments of the cognitive behavioral system 10 or methods, an introductory email may be sent to a user or group of users with voluntary opt-in and opt-out. All users or group of users opting-in are referred by sending their contacts to a secure portal and entered to a database. Each user or group of users opting-in receives a questionnaire and personal algorithm. The participant's personal algorithm becomes the motivational vehicle for performance-based biofeedback messaging as part of the machine and deep learning process.

In certain embodiments of the cognitive behavioral system 10 or methods, individual raw claims data may be sent to a secure portal and collated to a database. The data may be automatically and continuously stored and processed for deriving: a) individual and group financial cost profiles and b) clinical profiles according to specific diseases and claims. Clinical profiles may be segregated into a population health platform for some of the common ABCD costs for individual savings. All financial and clinical profiles may be updated on enterprise-level dashboards in real time.

Laboratory data may be individually entered to the clinical profile along with alerts.

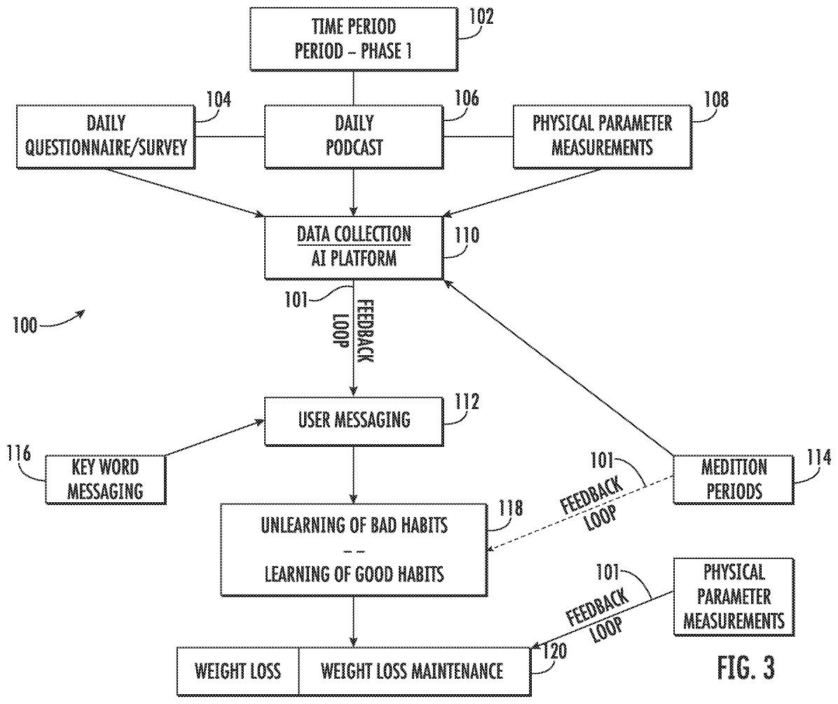

FIG. 3 outlines an illustrative example of an cognitive behavioral method for modifying subconscious brain habitualization relating to weight loss or weight loss maintenance cognitive method for modifying subconscious brain habitualization relating to weight loss or weight loss maintenance, referred to generally as weight loss method 100, using one or more components of the weight loss system 10. The weight loss method 100 is designed to help the user 12 to: 1) employ cognitive theory to permanently overcome old, unhealthy habits of lifestyle, behavior, and nutrition, 2) provide an automated clinical framework for monitoring and support, and 3) empower user 12 to develop the habit of employing multiple personal behavioral tools. Positive reinforcement may be achieved by: compliance, weight loss, medication discontinuation, physical parameter measurements, and meditation periods in which feedback loops 101 are employed and members may be asked to develop the habit of visualizing themselves at the best time of their life. For example, users may be asked to remember how they looked, felt, and thought at their best when they were at a more youthful, healthy self to which they might choose to return. An example of using negative reinforcement for subconscious remodeling could start each day's by standing naked in front of their early AM bathroom mirror, emotionally saying, “That's NOT me!”. Other images, such as a lazy boy chair, unhealthy food, or a TV set remote control, are provide further examples of employing negative subconscious remodeling for “That's not me!”.

In an illustrative embodiment of the weight loss method 100, the user 12 will undertake a Phase 1, 12-week program, see step 102 where machine learning from personal algorithms 25 and automated feedback messaging from clinical progress, patient reported quiz answers, and meditation form the building blocks users 12 employ during their active weight loss phase (Phase I), and Phase II, over the next 21-months of formal weight loss maintenance. The weight loss method 100 benefits users 12 by providing the user 12 mechanisms to learn how to easily lose and continue losing weight long term. On each day, the user 12 will answer a series of personal questions from data questionnaire(s) or survey(s) 42, e.g.; nicknames, or children's name, see step 104. User 12 will also view podcasts 44 daily, see step 106. Such podcasts may be, for example, seven minutes in length and have videos and audible text the user 12 can view or listen to. A user 12 completing (“completers”) at least twenty-five percent (25%) of their podcasts 44 and personal questions from the data questionnaire(s) or survey(s) 42 are believed to be motivated to adopt behavioral strategies, and believed to continue losing significant weight long-term. It is believed that completers may enter T2DM remission at a higher and faster rate than users that do not complete at least a portion, such as twenty-five percent (25%), of their therapy podcasts 44 and personal questions from data questionnaire(s) or survey(s) 42.

User 12 may also utilize the one or more health related physical parameters measurement devices 30 to obtain information related to the user's physical conditions status, see step 108.

The information obtained from the daily personal questions from the data questionnaire(s) or survey(s) 42 is sent to and stored in the one or more data storage and processing unit(s) 14, see step 110. Data obtained from the one or more health related physical parameters measurement devices 30 is also sent to and stored within the one or more data storage and processing unit(s) 14. The weight loss method 100, therefore, creates data sets of information in order to incentivize and measure compliance. The data from the one or more health related physical parameters measurement devices 30 can be used to create clinical data and clinical progress data.

Subconscious recalibration may therefore begin by user 12 answering the series of personal questions, after which the automated cognitive behavioral platform 24 and personal algorithms 25 use information, stored or real time, to motivate users 12 upon receiving the clinical data points, or dissuade users 12 from an action. For example, the automated cognitive behavioral platform 24 may send a message, see step 112, to user 12 via smart device 20, such as a personal congratulation. Based on the information received from the one or more health related physical parameters measurement devices 30, the automated cognitive behavioral platform 24 via the personal algorithms 25 may assist in subconscious recalibration by sending the user 12, via smart device 20, personal messages. For example, in order to habitualize the relationship of obesogenic meals, inactivities, and/or negative attitudes, participants are suggested to verbalize those events to themselves. For example, after arising, removing their bed clothes, and voiding, subjects are encouraged to look at themselves in a full mirror completely naked and shout, “That's not me!” Whenever participants see obesogenic meals, inactivities, and/or negative activities, Participants will receive personal messaging. For example, a) “Good AM . . . Name” or b) “Look in the mirror, naked, and repeat”, “That's not me” with emotion. Participants are encouraged to repeat this phrase numerous times during your day as the need arises in order to convince the subconscious of their new determination to lose weight and get healthy.”

Stress stimulates cortisol release from the adrenal glands, causing insulin resistance, increase blood sugar, and weight gain. Avoiding stressful situations by using “That's not me” and habitualizing regular periods of meditation for relaxation are helpful for stress reduction. The weight loss method 100 may include automated daily meditation messaging from quiz answers in order to reduce stress, increase focus and compliance, and increased weight loss and maintenance, see step 114. As user 12 is developing the habit of meditating, machine learning by biofeedback loops to record personal meditation elements most helpful for relaxation and reduce stress reduction, the user 12 may receive messages which help maximize the meditation period, such as, “Visualize your best shape and most relaxing place”.

Without subconscious modification, weight loss is challenging, and nearly impossible long term. The subconscious is the protector of the status quo, in this case, obesity caused by obesogenic habits, which must be broken for successful long-term weight loss. The weight loss method 100 encourages the participant to make a conscious effort to habitualize the use of key words or key phrases in order to use repetition to literally overwhelm the subconscious to re-learn the denial process for being able to avoid unhealthy choices. This process begins immediately upon arising after voiding, weighing-in, and going naked to a mirror to emotionally shout to the subconscious, “That's not me!”, see step 116, to send out personalized messages, such as “Hey, Jimmy, That's not me!”, or “Hey, Julie, Re-organize your brain!”, “Hi Mary, Stay positive!, “Hi Tom, Don't cut corners!”, “I will be my best!”, “Be prepared to win”, “Stay out of the kitchen after dinner”, “Your Mom, Robin, says Eat dinner before 7 PM and go to bed before 10 PM to lose maximal weight”, or “Your father, Frank, wants you to go for a relaxing walk after dinner”. Users/patients are encouraged to respond to these messages thereby adding to their deep learning profile.

The automated cognitive behavioral platform 24, via the personal algorithms 25, and Daily Therapy is based on information it receives, and may send user 12 the following messages, or similar type messages:

The basis of long-term success is subconscious recalibration.

“That's not me!”—repeat the key words.

Meditation: use a younger, healthier photo to “Visualize your success”.

“Thanks to growth hormone, most weight loss occurs at night, so go to sleep by 9:30 or 10.”

“Be your best.”

“Physical activity every day.”

“Water intake.”

“Eat healthy ketogenic meals.”

“Drive your blood ketones up with regular physical activity.”

“Weight loss progress”

“Medication discontinuation.”

“Hemoglobin alc: drive it below 6.5.”

“Test blood glucose levels at least 1×/day.”

“Stay out of the kitchen!”

“Be prepared to win!”

“Don't cut corners!”

“Healthy snacking!”

“Avoid rich, fatty foods!”

“Stay positive!”

“Avoid starvation!”

“Eat lots of greens!”

“Eat protein 3 times each day.”

“Eat a good protein breakfast.”

“Develop an anti-stress plan without your favorite comfort foods.”

“Plan how to eat out safely.”

The weight loss method 100 may employ user 12 selecting photos of challenging experiences related to, for example, a couch or Lazy Boy chair:

a) Triple mac and cheese

b) Taco Bell

c) “You do not need the Midnight kitchen raid, so go to bed early and avoid it.”

d) Bread and butter

e) Ice cream

f) Sodas

g) Alcohol

h) Cake and cookies

i) Fatty, fried foods

j) “Learn how to Keep your arteries, heart, brain, and organs healthy”.

The automated cognitive behavioral platform 24, using the personal algorithm 25, may be used to provide feedback based on task completions, response to clinical progress, and response to user compliance and motivation, i.e. congratulatory or non-congratulatory messages. As the cognitive behavioral system 10, via the feedback loops, i.e., one of the various mechanisms to capture and use data, in real time or captured, historical user data, and data processing software functionality of the AI platform 24 obtains data on the user 12, the weight loss method 100 may include a response of sending additional messages to the user 12 (preferably to the user's 12 computer 18 or smart device 20). Messages may be, for example, related to:

a) a reminder to do something;
b) a statement of encouragement;
c) a statement of admonishment.

The weight loss method 100 may include user 12 submission of visual pictures, such as a photo of the user 12 at a younger age for pre-meditation messaging. The weight loss method 100 may include the user 12 being messaged to tell their subconscious, “That's not me!” when they see various situations or environments, such as seeing themselves in a mirror or upon seeing unhealthy food or inactivities. The weight loss method 100 may allow the user 12 to respond to their mobile app's activity link by selecting the unhealthy choice. The automated cognitive behavioral platform 24 may be configured to affirmatively respond with a supportive, activity-specific message.

Each one of the steps, individually or in combination, are designed for subconscious reprogramming which includes the unlearning of old and established behavior that has lead the user 12 to engage in unhealthy habits as it relates to weight loss/gain and the leaning of new, healthy habits, see step 120, in order to allow the user 12 weight loss and weight loss maintenance, see step 120.

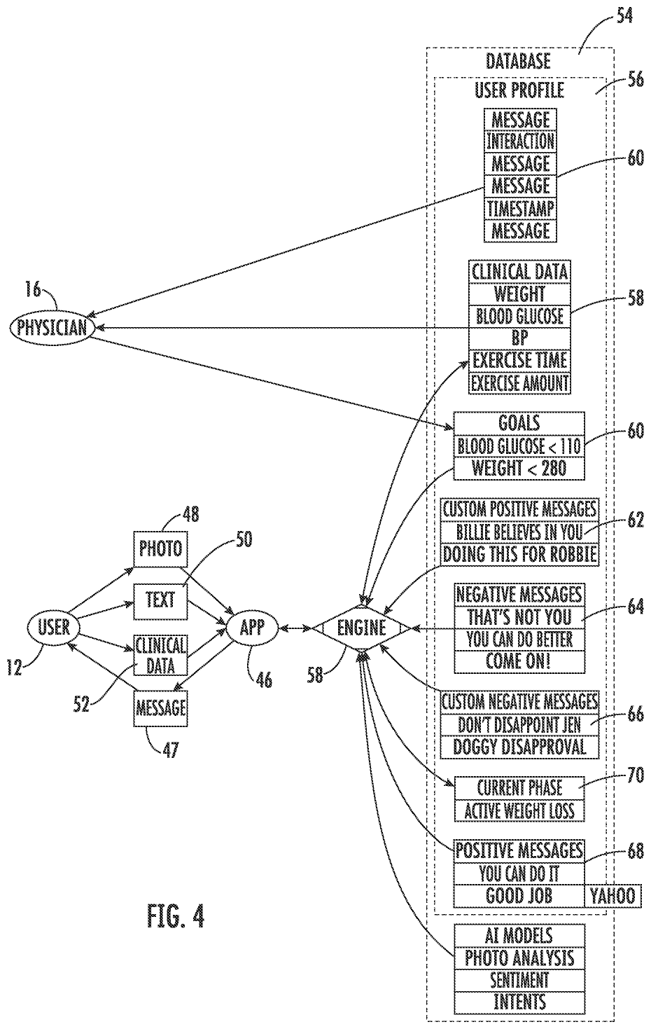

Referring to FIG. 4, the interactions of various components of the cognitive behavioral system 10 are shown. One or more of the individual components of the cognitive behavioral system 10 playing a role in the weight loss method 100. As illustrated, various components and activities of the cognitive behavioral system 10 are designed to interact with the patient/user 12 through software application 46 (on computer 18 or smart device 20). The interaction may be through data being input by the patent/user 12 or via messages 47 sent to the patient/user 12 via App 46. As shown, the patient/user 12 may provide information that may be processed via algorithms and/or artificial software programs via submitting photos 48, texts 50, or clinical data 52. Databases 54 may include information related to patient/user profile 56. The patient/user profile 56 incudes various information which can be used by artificial intelligence programing engines 58 to accomplish one or more functions described herein related to the cognitive behavioral system 10 and/or the weight loss method 100, including selection of reinforcement/feedback most likely to be effective in each situation, as well as enhancements such as extra reminders/reinforcement. The patient/user profile 56 may include information related to various messages 60 that may be set to the patient/user 12. The patient/user profile 56 may include information related to clinical data 58, including weight loss, weight loss %, blood glucose, blood pressure, exercise time, medication discontinuation. The patient/user profile 56 may include information related to user/patient goals 62, such as blood glucose being less than 125, BMI, or weight less than 280. The patient/user profile 56 may include one or more custom positive messages 62, negative messages 64, custom negative messages 66, or positive messages 68. The patient/user profile 56 may include information related to current phase, 70. The patient/user profile 56 may include information related artificial intelligence modeling. One or more of the information within databases 54 by the physician 16 for the purpose. The patient/user profile contains all of the information for interactions, results, and compliance for a particular patient. This information is used for many purposes, including evaluation of the patients' progress and compliance, as well as information used to train the artificial intelligence (AI) engine to determine which interactions are most effective in different situations. The Physician is responsible for the patient's health and overall progress, and requires access to this data in order to make their determinations and recommendations.

Example Usage: Weight loss or weight loss maintenance for long-term virtual remission of type 2 diabetes (T2DM)

A patient/user uses the cognitive behavioral system 10 and methods in accordance with embodiments of the invention for weight loss or weight loss maintenance purposes. The cognitive behavioral system 10 and methods in accordance with embodiments of the invention utilizes intelligent automation, including a combination of AI and automation supported by data modeling for effective achievement of goals, i.e. in this use, weight loss or weight loss maintenance for those suffering type 2 diabetes (T2DM). The subconscious brain works to maintain the status quo of obesogenic habits, preventing new, healthy habit formation, or modification of old unhealthy habits. The cognitive behavioral system 10 and methods in accordance with embodiments of the invention is configured to help patients/users learn how to break old, bad obesogenic habits and install new, healthy habits for long-term health through a combination of personal, self-learning algorithms to reinforce cognitive behavioral therapy (CBT): nutrition, physical activity, and lifestyle measures.

The cognitive behavioral system 10 and methods in accordance with embodiments of the invention apply cognitive artificial intelligence (AI) to CBT in order to recalibrate the subconscious to automate, scale, and improve upon our non-AI clinical result, such as for long-term virtual remission of type diabetes (T2DM). The weight loss system and methods in accordance with embodiments of the invention helps patients lose weight transiently by employing only one (1) or two (2) CBT components. However, all three (3) CBT modalities must be simultaneously employed for optimal, long-term weight loss and T2DM remission.

The sentinel, unlearning event in the subconscious of the obese patient occurs the moment of weight loss success visualization in which all 3 CBT modalities converge to help produce noticeable weight loss validation during weight loss weeks 1 of 12. From this sentinel, “I got it”, “That's me”, or “Yahoo” moment, the patient/user will receive daily motivational biofeedback messaging from his/her personal algorithm in order to quantify and magnify the importance of the sentinel moment.

The cognitive behavioral system 10 and methods in accordance with embodiments of the invention may also provide for spontaneous personal self-talk messaging numerous times daily. e.g.: Hi, nickname, Did you use your “I got it”, That's me“, or Yahoo” weight loss moment today? Yes or No response required.

The cognitive behavioral system 10 and methods in accordance with embodiments of the invention may also be configured to collect and feedback patient's/user's daily conscious weight loss success visualization, “That's me!” or “That's not me!”, for influencing their subconscious decision making, e.g.: Hi, nickname, Did you use your “That's me!” or “That's not me!” today? Y N.

To start, patients/users are asked to volunteer simple answers to personal questions. Responses to these questions will be used to build a “dynamic, self-learning, personal algorithm” in order to increase interest, motivation, and compliance after receiving routine messaging and daily clinical progress reports.

Positive and negative messages, along with personalized of them, may be initially chosen randomly to be sent to the patient/user. A weighting algorithm may be used to avoid sending the same message too frequently. The cognitive behavioral system 10 and methods in accordance with embodiments of the invention may be configured to measure the effectiveness of the messages by monitoring clinical data, compliance, and any direct feedback from the patient/user. The effectiveness of the messages will be use to “learn” which of the messages and personalization's have the most impact on the varying situations, i.e. after a food binge, when a patient/user is doing well, before and after competency tests, for each individual patient/user. This is “adaptive intelligence” on the way to “Imprinting” the subconscious.

The cognitive behavioral system 10 and methods in accordance with embodiments of the invention may utilize two models, one aggregate set to identify individual situations (segmentation) and one for the messages themselves (a set of liner regressions). The aggregate models are designed to take advantage of the greater amount of data across multiple patients/users to get a corpus of useful size quickly, and average out individual variations. The second set of models may be individual models, three per patient/user: two for the situations and messages, and one for the personalizations. The personalization model will necessarily only exist per-patient/user, as each patients'/users' personalizations and preferences for them will be different. The situation and message models may be designed to work like the aggregate ones, but offer fine tuning for individual patients/users and their situations.

As the models gain training data and predictive ability, they can be leveraged to take an increasing role in choosing messages and personalizations instead of the original, mostly random approach. The models may be employed by feeding in the current and recent data to the situation models, to identify broad trends (with the aggregate model) and personal idiosyncrasies (with the personal model). The output of the model, along with physician input, phase of the process, and calendar date, will be used to identify the patient's/user's current likely situation. Given the situation, clinical data, phase, etc., the message models may be configured to assign weights to the various available choices, based on which ones have historically done well in this situation previously. A choice of message may be made based on these weights, then the personalization model will be used to choose which patient-specific personalization(s) to apply to it, and the chosen personalized message will be sent to the patient.

As an illustrative example, the patient/user will be sent a text message to his/her electronic device, i.e. a smart phone, a computer tablet, smart watch, sometime(s) during the day, preferably each morning and afternoon for a time period, such as 52 weeks:

Various data will be recorded and messaged. The data may be recorded by the patient/user based on simple responses to questions, or by obtaining physical parameter measurements, i.e. using a scale to obtain patient/user weight, ECG reading, blood glucose levels. For example:

1. “Daily Therapy” quiz answer submission (submitted to patient/user by system/app)

2. Daily weight (entered by system)

3. Total weight loss (W-L) %

4. Total weight loss (W-L) (lbs)

5. Eating each of 6 meals/day and time within 3-4 hours (patient recorded)

6. Drinking at least 6-8 glasses of water/day (patient recorded)

7. Blood glucose (system recorded 2×/day)

8. Blood ketones (system 2×/week)

9. Time to sleep (system)? The system records how long was slept.

10. Daily physical activity (system)

11. Daily meditation (member recorded) Y N

12. End of day total insulin dosage (member recorded)

13. Date insulin discontinued (by member)

14. Number of days in T2DM remission (member recorded)

15. Stress testing by social exposure to obesogenic situations. (member recorded) In this interaction, the user/patient records the number and type of obesogenic situations and receives a congratulatory personalized message.

For the W-L results, the algorithm may be configured to provided an alert, personal congrats and message, i.e. “That's me” for weekly W-L>1%.

Compliance monitoring: Results of clinical and patient recorded data may be entered individually and collectively by day and week as percent (%) of compliance. Alerts may be sent based on predetermined percent obtained. For example, keeping compliance high (80% participation) is key to successful long term weight loss. Accordingly, alerts may be send according to <80% target compliance.

Algorithm Training:

Patients/Users may be asked to take a picture or answer a personal question. For example, Negative messages: (“that's not me!” . . . Repeat with a clinched first when you see this to break a bad habit!). This message is attached to all the following 1× per day. The image could be, for example, of mac and cheese or chocolate cake.

By continually providing the message, the patient's/user's subconscious will be constantly targeted order to associate “That's not me!” to their status quo unhealthy habits of lifestyle, behavior, and nutrition until the status quo changes.

1. If A: See picture of themselves naked in a mirror. Then B (That's NOT ME!) message is provided

Every morning, a picture is sent, the picture including a first to remind the patient/user to see themselves in a mirror naked in a fighting stance with their first to show determination in order to break the status quo acceptance of obesity of their subconscious brain.

a) All the below situations and responses are entered to an algorithm.

e,# Then the below situation arrives, patient/user clicks the item daily as necessary and receives the response: “That not me and here's my fighting fist”.

A message, “Did it help?” Yes or No can be sent to the patient/user.

2. Picture of favorite fatty food and drinks sent

3. Picture of clock after 10 PM sent

4. Picture of old tight pants or belt sent, with message “I want this gone forever!”

5. Picture of elevator sent

6. Picture of TV remote control sent, with message, “Take extra steps! Make yourself an in-efficiency expert”

7. Picture of parking space near grocery entrance (as above)

8. Picture of patent/user Insulin bottle and syringe sent, with message “I want this gone forever!”

Personal algorithm positive messages: “That's me!” . . . repeat to strengthen a good habit!!!”

1. Photo of patent/user in their 20's-30's at their best, with message conveying to the patient/user, this is the time and place to which you can return.

2. Your Nickname (answer a personal question)

3. Your Favorite or personal name (answer)

4. 2 cups of cooked broccoli or greens is delicious

5. Bicycle—picture

6. walking shoes—picture

7. swimming pool—picture

8. pic of walking with mate or a dog

9. pic of a tree-lined street

10. scale: “be accountable”

11. physical activity and sleep watch

12. Most influential parent or family member

13. Photo of walking up steps

In use:

1. patients/users are first sent an introductory email.

2. A follow-up email describing the benefits of the program and asking type 2 diabetes patients/users if they're interested in learning about remission of their diabetes and associated diseases may also be sent.

3. The patient/users will be asked: Are you a type 2 diabetic? Yes? No?

4. The patient/users will be asked: Are you interested in knowing more about how we can help you to learn to help your type 2 diabetes to enter, stay in remission, and become healthy? Yes or No.

5. The patient/users will be asked: a) We need to be able to identify ideal candidates for diabetes remission. Are you willing to approve our fully private review of your healthcare claims data under Federal HIPAA guidelines? Yes? If so, that's great! We have few basic questions as follows: (If not, thank you for your time.)

The patient/users will be asked: b) Are you using insulin? Yes or No.

The patient/users will be asked: c) Is your blood glucose generally controlled (at or below 130)? Yes or No.

d) If yes, see #14 below.

7. The patient/users will be asked: tell us your approximate height and weight. (we need to derive and record BMI).

8. The patient/user will be asked: Do you have access to a computer and can you use it? Yes or No.

9. The patient/users will be asked: Are you able to walk or swim for at least 30 minutes daily.

If all 1-8 are A, all patient/users will be sent a follow-up letter containing information for uploading to their personal algorithm:

All 1-9 is yes (A):

Any 9-12 is no (B):

9. The patient/users will be asked: Do you have any of these conditions?:

i. Pregnancy or planning to be pregnant within the next year

ii. end-stage health issues

iii. a heart attack within 6 months?

iv. Out of control blood glucoses on insulin

v. Hemodialysis

vi. Type 1 diabetes

vii. Chronic alcoholism or current opiate addiction

10. A list of potential interested patients/users requesting claims data for last 12 months to be sent to a portal.

11. Claims data sent to InterSystems, Inc. software portal for financial and clinical profiles.

12. Clinical profiles stratify to diabetes, high blood pressure, cardiovascular disease, peripheral neuropathy, peripheral vascular disease, retinal vascular disease, heart failure, obesity, COPD, male/female, hemodialysis, end-stage kidney disease, non-alcoholic fatty liver disease, chronic alcoholism, heart attack within 6 months, ability to walk or exercise at least 30 minutes daily, no intention to become pregnant for at least 1 year,

13. If 9-13 is A, send email of thanks for their information

14. If 9-13 is B: send enrollment invitation.

PERSONAL ALGORITHM 1: Patient/user requested to send for baseline:

1. Name: ______ Personal messages: (for progress)

2. Nickname or favorite name

System sends message: “Hi, ‘nickname’”

3. Member number:

4. Date of Birth

System sends message: “Hi, ______. This is ______. Remember, “Age is just a number!”

5. Gender:

6. Starting weight in pounds

7. W-L %

System sends message: “Hi, ______ After losing 3-5% weight, Your friends will say you look 20 years younger. Several times daily, congratulate yourself and tell your brain, “HOORAY for ME!!! I'm starting to look and feel great. I vow to keep going to 20% weight loss and stay there!”

8. Height in inches.

9. Body Mass Index (BMI)—system to calculate based on information inputted.

10. Type 2 diabetes history? Yes or No

11. Diabetic retinopathy? (blindness from diabetes) Yes or No

12. Diabetic kidney disease? Yes or No

13. High Blood Pressure? Yes or No

14. Heart disease? Yes or No

15. High cholesterol? Yes or No

16. COPD? Yes or No

Shortness of breath? Yes or No

18. How many flights of stairs can you comfortably walk?

19. Are you injecting insulin? Yes or No

20. Is your AM blood glucose generally controlled between 95 to 130? Yes or No

21. Number of blood pressure meds:

22. Bariatric Surgery? Yes or No

23. How far can you walk?

24. Do you suffer from depression? Yes or No

25. Do you have fatty liver? Yes or No

26. Are you sleeping less than 7-8 hours? Yes or No

27. Peripheral neuropathy? (Burning, pain & numbness in your feet? Yes or No

28. Do you suffer from erectile dysfunction? Yes or No

29. Do you have Peripheral vascular disease? Yes or No

30. Do you have hip, knee, or back pain? Yes or No

31. Number of times you went to the ER in last 6 months: 0, 1, 2, more.

MOTIVATION DATABASE: (link 1-9 with clinical progress data 1×/week).

1. What's the name of your best motivator?

2. Rank your Highest motivation for diabetes remission?

3. Sleep better

4. Quality of life

5. Live longer

6. Save money $$

7. Fewer meds

8. A smiling photo of yourself in a fighting stance with a clinched first showing determination.

9. Photo of family

ALGORITHM 2—Activities Before, during, and after reaching goal weight: SEND DAILY REMINDER

Please make entries in your Daily Journal

If A, then B response.

1. Asks, visualize yourself at your best? System requests a picture thereof.

2. Asks, what color is your favorite outfit? System reminds the patient/user to please visualize yourself

3. Asks, what was your belt size in your 20's inches. System reminds the patient/user to measure weekly.

4. Asks, what is your favorite physical activity? System reminds the patient/user to start with walking 15 minutes daily

Asks, what is your favorite sports team. Systems may add the logo

Asks, what is your favorite place in the world? Systems may send pictures of the place.

ACCOUNTABILITY: The weight loss system and methods in accordance with embodiments of the invention is configured to write and check off all goals & activities in the patient/user daily Journal; and send daily message reminder so patient/user members can easily comply and link to a motivator or “That's me!” message.

1. Asks: Are you holding yourself accountable? Yes or No, Link to “That's me!”

2. Asks: Weigh yourself every AM and did you lose 0.5-1.5 pound/day, Yes or No, (link, add logo of favorite sports team)

3. Asks: Make a first and shout “That's not me” at your naked self, Yes or No, look in the mirror and all day long with off limits activities or foods; show photo of clinched fist.

4. Asks: Eat a good protein breakfast. Yes or No, (Link)

5. Asks: List and check off all 6 meals/day and times. Confirm, Yes or No.

6. Asks: Eat 4 cups of green veggies/day? Yes or No.

Asks: Read and submit Daily Therapy and quizzes answers M-F? Yes or No. Reminds patient/user not to fall behind and feel the need to submit more than 1 quiz answers/day! Just 1 set of answers is perfect.)

7. Asks: Start daily physical activity on Saturday of your 1st week at 15 minutes/day. Slowly increase to 45 minutes by the end of week 2. Yes or No.

8. Asks: Eat your big protein dinner no later than 6 PM. Yes or No.

9. Eat a 6th protein shake snack before bed.

Yes or No.

10. Asks: Turn off your brain and cell phone and go to bed by 9 PM!!! Yes or No.

11. Asks: Stay out of the kitchen after dinner! Yes or No.

12. Asks: have you cheated, for example by eating snacks or the wrong snacks? It will slow your weight loss and remission. Yes or No.

13. Asks: Test your blood glucoses daily yes or no

The system may ask the patient/user to send Insulin doses.

14. Asks: Test your blood ketones weekly? Yes or no

Asks: Water: Are you drinking at least 8 glasses/day? Yes or No.

15. Asks: Frequency of meals: Are you eating protein at least every 2½-3 hours? Yes or No

16. Asks Daily Meditation. Yes or No

Algorithm 3: Clinical results. The weight loss system and methods in accordance with embodiments of the invention is configured to send reports to the patient/user or patient/user's physician weekly.

1. Insulin dose: Date ______ ______ units (member fill in)

Negative messages sent: (“that's not me!” . . . Repeat with a clinched first when you see this to break a bad habit!) We will attach this message to all the following items 1× per day.

Positive Messages: “That's ME”, upon reaching a goal.

2. HbAlc: ______ (member reports)

3. Physical activity ______ minutes (system reports)

4. Sleep ______ hours (system reports)

5. Initial Weight: ______ pounds (system)

6. Current weight ______ pounds (system)

7. Initial BMI: ______

8. Current BMI:

9. W-L: ______ pounds

10. W-L % ______ %

11. Goal Weight % 20% (That's me!)

12. YAHOO! Remission date (date off insulin and analogues) ______ (member reports); REMISSION Predictive Modeling Module

13. Remission days (If days off insulin and analogues) That's ME!

14. Compliance (Quiz answer submitted daily M-F): Yes or No

Connect the fowling entries with the above results:

Every AM, the weight loss system and methods in accordance with embodiments of the invention is configured to sends message: “That's not me!” and a first to remind member to see themselves in a mirror naked in a fighting stance.

16. Picture of favorite fatty, sweet, or fried food and drinks.

17. Picture of clock at 10 PM

18. Picture of old tight pants or belt

19. Picture of elevator

20. Picture of TV remote control

21. Picture of parking space near grocery entrance

22. Picture of patient/user Insulin bottle and syringe

ALGORITHM 4: Positive messages:

(That's me!” . . . repeat to strengthen a good habit!!!” (link)

1. Photo of you in your 20's-30's at your best.

2. Your Nickname (answer a personal question)

3. Your Favorite or personal name (answer)

4. 3-4 cups of cooked broccoli or greens with onions and garlic is delicious

5. Picture of Bicycle

6. Picture of walking shoes

7. Picture of swimming pool

8. Picture of walking with mate or a dog: “Enter diabetes remission to become more independent!”

9. Picture of a tree-lined street

10. scale: “be accountable” physical activity and sleep watch “Sleep earlier to sleep better”

11. Spouse's name

12. Favorite activity

13. Favorite child's or grandchild's name

14. Mother's name

15. Father's name

16. Pic of family

Algorithm 5: Health Coaching and Slow Weight Loss Improvement tips: The cognitive behavioral system 10 and methods in accordance with embodiments of the invention is configured to send messages such as:

Hi, nickname, your expected daily weight loss is 0.5-1.5 pound per day. There are many reason why you could be a slow weight loser:

1. “Hi, nickname, your're losing ______ pounds/day, which is a little less than your best.”

2. “Hi, nickname, to lose the fastest, please eat all 1 of your 6 protein meals every 3-4 hours to avoid slowing your metabolism by starvation, binge eating.”

3. “Hi, Nickname, avoid the kitchen after dinner?”

4. “Hi nickname, high water intake is the secret of weight loss! Please drink all 6-8 glasses of water/day.”

5. “Hi, nickname. Please eat your big evening meal (including all veggies) in the middle of the day.”

6. “Hi, nickname, your daily physical activity is ______. Remember to develop the good habit of at least 45 minutes daily physical activity to increase your metabolism and feel your best.”

7. “Hi nickname, Please make it a habit to always eat a good protein breakfast immediately upon arising.”

9. “Hi nickname, please avoid extra carbs, alcohol, and fruit.”

10. “Hi nickname, your average sleep is ______. Please develop the habit of going to bed no later than 9:30 PM to give your brain's growth hormone a chance to burn your fat?”

11. “Hi Nickname. Insulin makes and keeps you overweight, so please safely wean down your insulin and analogues to normalize your overweight as fast as possible.”

12. “Hi nickname, Stress plays a role in weight gain and high glucose levels. Remember to meditate several minutes every day to relieve stress, help lose weight, improve your mood, and enter diabetes remission.”

13. “I'm generally meditating ______ minutes/day. (If 5-10 minutes>>>>BRAVO. If less, >>>>>your (top motivator) wants you to do better!”

14. “Hi Nickname. Blood ketones measures the rate of your muscles to burn your fat. Keeping your physical activity at least 45 minutes/day results in high blood ketones, rapid weight loss, and diabetes remission. Your blood ketones started at ______ and now range from ______”.

Algorithm 6: Meditation. The weight loss system and methods in accordance with embodiments of the invention is configured to send messages such as:

1. “Hi, nickname, Are you making time to meditate daily? Yes or No”

2. “Is it helping you to relax, focus on staying positive, and on track? Yes or No.”

Algorithm 7: Satisfaction (separate outcome results)

The weight loss system and methods in accordance with embodiments of the invention is configured to send messages such as:

1. “Are you satisfied with this program so far? Yes or No”

2. “Are you learning new habits and breaking old ones? Yes or No”

3. “Are you feeling better yet? Yes or No”

4. (Your SPOUSE NAME approves your progress) “Are you starting to feel more independent and in control? Yes or No”

5. Nutrition:

a. “Does the nutrition satisfy you? Yes or No”

b. “Are the nutrition tips helpful? Yes or No”

6. “Are you sleeping better yet? Yes or No”

7. “Are you aware your new subconscious is helping you to make healthy choices? Yes or No”

If Yes, “congrats, ______ nickname”

ALGORITHM 8 (Monthly follow-up) Weight loss contributes to clinical resolution of all these conditions. The weight loss system and methods in accordance with embodiments of the invention is configured to send messages such as:

1. Your favorite child or grand child ______:

“After ______% weight loss, are you feeling better? Yes or No

Message sent: No, Start h=Here . . . . Yes . . . “Congrats Nickname, Close loop

2. Diabetic kidney disease? Improved? Yes or No.

3. High blood pressure? Are you off some meds? Yes or No.

4. Shortness of breath, improved Yes or No.

Answer Yes, message sent: “Congrats Nickname”

5. Best friend ______: “How many flights of stairs can you comfortably walk?”

0—Start the loop here

1 flight

2 flight

3 flight

4 flights—close loop here

6. Congrats!!! Nickname. We see you're reducing or off insulin since ______. No, Start loop here, Yes, Close loop here

7. Number blood pressure mediations off? 0 (start), 1, 2, 3 (“Congrats Nickname”

8. How far can you walk? Better, 4 blocks?, 3 blocks?, 2 blocks?, 1 block, further than 4 blocks.

9. Depression? Better, Yes or No

10. Fatty Liver? Better, Yes or No

11. Trouble sleeping 7-8 hours? Better, Better, Yes or

No

12. Peripheral neuropathy? Better, (Burning pain numbness) Better, Yes or No

13. Erectile Dysfunction (ED) improve? Better, Yes or

Message sent: “Name of your partner, says thanks!”

14. PVD? (Peripheral vascular disease) Better, Yes or No

Hip, knee, or back pain reduced? Yes or No

“Nickname,” Number of times you went to the ER during last 6 months last year?

Once completed, a message of: “Congrats, ‘nickname’!”.

ALGORITHM 9: DATABASE

Based on the information learned from user input, the weight loss system and methods in accordance with embodiments of the invention is configured to send messages such as:

1. “Good morning ______. Your sleep monitor and your belt size will soon be showing how important a good night's sleep is. Congrats! Going to bed by 10 PM! (spouse) will be proud!!!”

2. “Hi ______. Great news on completing your quizzes! Your (favorite child) will be happy for you!!!”

3. “Hey ______. Glad your weight loss is going in the right direction. Your (family) will be proud!”

4. “Good day ______. Congrats on using “That's not me”! to lock in your new habits. Your family will be happy for you!!!”, with a picture of the patent/user.

5. “YAHOO! ______. Congrats on decreasing your insulin requirements!”

6. “Hi ______. Your ______ will be happy about your high water intake.”

7. “Hi ______. All your family will approve of your weight loss by the new habit of bedtime before 9 PM.”

8. “Congrats! You blood ketone level of ______ optimally 0.5-3.0 mg/dL shows your physical activity level of ______ and ketogenic nutrition is consistent with fat burning and a trend to diabetes remission.”

9. “Congrats on meditating daily. Your decreasing waistline and relaxed inner spirit are appreciative.”

10. “Hi ______. You're heading to diabetes remission and independence.”

Algorithm 10: T2DM Remission: Predictive Modeling based upon % Weight Loss. Based on the information learned from user input, the weight loss system and methods in accordance with embodiments of the invention is configured to send messages such as:

1. “Hi ______. After ______ (for example 12) of 60 program weeks, your % weight loss is %. From Program Day 1, your weight loss will start helping your pancreas, liver, and total body to reduce their fat content to start the process of diabetes remission. To ensure remission, off insulin, and analogues, we ask you to aim for 20% weight loss after 12 weeks. You can expect your insulin requirements to be zero starting at an average 8% weight loss within 5.2-5.3 weeks or sooner. You can expect to continue receiving, “That's me!”, progress messages because we want you to continue your success for the next 12 months and beyond.”

2. “Hi, ______. Compliance in completing only one (1) Daily Therapy podcast and submitting Quiz Answers is the key to Diabetes Remission. Perfect compliance of completing 60 podcasts over 12 weeks is number completed/number of weeks. So, your perfect 1st weekly compliance is 5/5=100%. You submitted ______ podcasts out of a possible ______, so your compliance=______ or %______.”

Algorithm 11: Testing Subconscious Remodeling. Based on the information learned from user input, the weight loss system and methods in accordance with embodiments of the invention is configured to send messages such as:

1. If A: How many times per day are you using keywords “YAHOO”, “That's me” or “That's not me” for new habit formation?

a. 1-3

b 4-6

c. more than 6

Then B: send “CONGRATS” message if a, b, or c

2. If A: Is this helpful for new habit formation? Yes or No

Then B: Send “Congrats” if Y.

3. If A: Is the convenience of the scale, activity monitor, sleep monitor, and blood ketone meter helping you to form new habits? Y N

Then B: If Yes, then send “Congrats” message

4. If A: Do the personal feedback messages help motivate you? Yes or No

Then B: If Yes, then send “Congrats” message.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A cognitive behavioral system for modifying subconscious brain habitualization relating to weight loss or weight loss maintenance comprising:
an electronic device having a platform/software program that implements a cognitive behavioral therapy algorithm that utilizes various data about a user in need of modifying subconscious brain habitualization relating to weight loss or weight loss maintenance;
one or more databases for receiving and storing data sets related to said user in need of modifying subconscious brain habitualization relating to weight loss or weight loss maintenance.

2. The cognitive behavioral system for modifying subconscious brain habitualization relating to weight loss or weight loss maintenance according to claim 1, wherein said electronic device having a platform/software program is configured to provide various feedback applications.

3. The cognitive behavioral system for modifying subconscious brain habitualization relating to weight loss or weight loss maintenance according to claim 4, wherein said feedback is in the form of personalized user messages relating to task completions, response to clinical progress, motivation, or combinations thereof.

4. The cognitive behavioral system for modifying subconscious brain habitualization relating to weight loss or weight loss maintenance according to claim 1, further including one or more physical parameters measurement devices designed to monitor, measure, and obtain data associated with one or more health related physical attributes of said user in need of modifying subconscious brain habitualization relating to weight loss or weight loss maintenance.

5. The cognitive behavioral system for modifying subconscious brain habitualization relating to weight loss or weight loss maintenance according to claim 4, further including one or more physical parameters measurement devices include a blood glucometer, a blood ketone meter, a physical activity monitor, a sleep monitor, a scale, a cardiac monitor, or combinations thereof.

6. The cognitive behavioral system for modifying subconscious brain habitualization relating to weight loss or weight loss maintenance according to claim 1, further including artificial intelligent (AI) system configured to process collected data relating to said user in need of modifying subconscious brain habitualization relating to weight loss or weight loss maintenance.

7. The cognitive behavioral system for modifying subconscious brain habitualization relating to weight loss or weight loss maintenance according to claim 6, wherein said AI system is configured to analyzes data sets and groups items together based on association and similarity to identify clusters of user behavior predictive of said user success.

8. The cognitive behavioral system for modifying subconscious brain habitualization relating to weight loss or weight loss maintenance according to claim 1, further including messaging based on one or more actions taken by said user in need of modifying subconscious brain habitualization relating to weight loss or weight loss maintenance, said messaging including written messages, pictures, or combinations thereof.

9. The cognitive behavioral system for modifying subconscious brain habitualization relating to weight loss or weight loss maintenance according to claim 1, further including use of one or more therapy and quiz sessions.

10. The cognitive behavioral system for modifying subconscious brain habitualization relating to weight loss or weight loss maintenance according to claim 1, further including use of one or more video podcasts.

11. A method of electronically modifying subconscious brain habitualization for achieving weight loss or weight loss maintenance, comprising:
electronically obtaining data from a user in need of modifying subconscious brain habitualization relating to weight loss or weight loss maintenance;
based on said data obtained, providing said user in need of modifying subconscious brain habitualization relating to weight loss or weight loss maintenance, one or more messages to aid in weight loss or weight loss maintenance;

12. The method of electronically modifying subconscious brain habitualization for achieving weight loss or weight loss maintenance according to claim 11, wherein the type of message sent is generated using an artificial intelligence based network configured to send said messages images based on user response.

13. The method of electronically modifying subconscious brain habitualization for achieving weight loss or weight loss maintenance according to claim 11, wherein said messages are in the form of a text, images, or combinations thereof.

14. The method of electronically modifying subconscious brain habitualization for achieving weight loss or weight loss maintenance according to claim 11, wherein said data relating to said user is obtained through the use of daily questionnaires, podcasts, or combinations thereof.

15. The method of electronically modifying subconscious brain habitualization for achieving weight loss or weight loss maintenance according to claim 11, further including the step of:
obtaining physical parameter measurement data of said user in need of modifying subconscious brain habitualization relating to weight loss or weight loss maintenance; and
based on said physical parameter measurement data obtained, providing said user in need of modifying subconscious brain habitualization relating to weight loss or weight loss maintenance with one or more messages.

16. The method of electronically modifying subconscious brain habitualization for achieving weight loss or weight loss maintenance according to claim 15, wherein said physical parameter measurement data is obtained from a blood glucometer, a blood ketone meter, a physical activity monitor, a sleep monitor, a scale, a cardiac monitor, or combinations thereof.

17. A non-transitory computer readable medium having computer readable instructions embodied thereon for modifying subconscious brain habitualization for achieving weight loss or weight loss maintenance, wherein, when executed by at least one processor of an electronic device used by an individual need of modifying subconscious brain habitualization relating to weight loss or weight loss maintenance with one or more messages, the computer-executable instructions cause at least one processor to at least perform operations comprising:
electronically obtaining data from said individual in need of modifying subconscious brain habitualization relating to weight loss or weight loss maintenance;
based on said data obtained, providing said individual in need of modifying subconscious brain habitualization relating to weight loss or weight loss maintenance, one or more messages to aid in weight loss or weight loss maintenance.

18. The non-transitory computer readable medium according to claim 17, wherein said at least one processor further performing operations comprising:
obtaining physical parameter measurement data of said individual in need of modifying subconscious brain habitualization relating to weight loss or weight loss maintenance; and
based on said physical parameter measurement data obtained, providing said individual in need of modifying subconscious brain habitualization relating to weight loss or weight loss maintenance with one or more messages.
